(19) United States
(12) Patent Application Publication
(10) Pub. No.: US 2004/0253332 A1
(43) Pub. Date: Dec. 16, 2004

(54) Method for regulating the appearance of skin containing combination of skin care actives

(76) Inventor: Ratan K. Chaudhuri, Lincoln Park, NJ (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/803,160

(22) Filed: Mar. 18, 2004

Related U.S. Application Data
(63) Continuation-in-part of application No. 10/120,156, filed on Apr. 11, 2002, now Pat. No. 6,649,150.
(63) Continuation-in-part of application No. 10/616,299, filed on Jul. 10, 2003.
(60) Provisional application No. 60/455,396, filed on Mar. 18, 2003.
(60) Provisional application No. 60/395,612, filed on Jul. 15, 2002.

(51) Int. Cl. A61K 35/78; A61K 7/06; A61K 31/7048; A61K 31/7008

(52) U.S. Cl. 424/775; 514/27; 514/62; 424/74; 514/356; 514/559; 514/725

(57) ABSTRACT

A method for regulating the appearance of skin comprising topically applying to said skin a composition comprising: (a) a cosmetically or pharmaceutically acceptable carrier and about 0.05% to about 5% of an extract comprising a low molecular weight hydrolysable tannins, and mixtures thereof; (b) an effective amount of at least one additional skin care active ingredient selected from the group consisting of ant-acne actives, retinoids, ant-cellulite agents, antimicrobial actives, antifungal agents, vitamins, anti-inflamatory agents, tanning agents, allantoin, glucosamine, phytantriol, hydroxyacids, niacinamide, phytosterols, sunscreens and mixtures thereof.

### CROSS-REFERENCE TO RELATED APPLICATION

This application claims the benefit of Provisional Application 60/455,396 filed Mar. 18, 2003 and is a continuation-in-part of application Ser. No. 10/120,156 filed Apr. 11, 2002, now U.S. Pat. No. 6,649,150 B2 entitled “Skin Lightening” issued Nov. 18, 2003 and of regular application Ser. No. 10/616,299 filed Jul. 10, 2003 entitled “An Effective Method For Regulating The Appearance Of Skin” claiming the benefit of Provisional Application 60/395,612 filed Jul. 15, 2002. It is also related to regular application Ser. No. 10/660,742 filed Sep. 12, 2003 entitled “Process For Enhancing Bioactive Principles Of Phyllanthus Emblica Extract” and Ser. No. 10/616,494 filed Jul. 10, 2003 entitled “Topical Anhydrous Delivery System” and all the provisional applications mentioned in the regular applications and patents.

### FIELD OF THE INVENTION

This invention relates to novel methods for regulating the appearance of the skin by using a standardized extract of Phyllanthus emblica and other skin care actives to reduce premature skin aging.

### BACKGROUND OF THE INVENTION

Photo aging is mainly due to ultraviolet radiation of sunlight, which overwhelmingly contributes to premature aging even in young adults. As skin is increasingly exposed to ultraviolet (UV) radiation, the risk for photo-oxidative damage to skin with long term detrimental effects, characterized by wrinkles, fine lines, and loss of skin tone and resilience increases. Photo aged skin displays alterations in cellular components and in the extracellular matrix characterized by i) the accumulation of disorganized elastin and its microfibrillar component fibrillin in the deep dermis and ii) severe loss of interstitial collagens, the major structural proteins of the dermal connective tissue. One of the pathogenic agents responsible for these changes is UV-induced reactive oxygen species (ROS), which deplete and damage non-enzymatic and enzymatic antioxidant defense systems of the skin. Photo aging of the skin is a complex biological process affecting various layers of the skin with major damage seen in the connective tissue of the dermis. The dermis lies below the epidermis and in conjunction with the basement membrane at the dermal-epidermal junction provides mechanical support for the outer protective layers of the epidermis. Any damage to the dermal components is seen predominantly on the sun-exposed body areas, especially on the face.

### UV-Induced Major Chemical and Biochemical Changes and Their Implications

Generation of Superoxide, Singlet Oxygen and Hydrogen Peroxide

There is now ample evidence that shows ROS generated in vitro and in vivo after UV-A and UV-B irradiation causes serious damage to skin (Jurkiewicz and Buettner, 1994). Besides direct absorption of UV-B photons by DNA and subsequent structural changes, generation of ROS following irradiation with UV-A and UV-B requires the absorption of photons by endogenous photosensitizing molecules. Recently, the identification of the epidermal UV-A absorbing chromophore transuroconic acid that quantitatively accounts for the action spectrum of photo aging, has been reported (Hanson and Simon, 1998). The excited photosensitizer subsequently reacts with oxygen resulting in the generation of ROS including superoxide anion and singlet oxygen. Superoxide anion and singlet oxygen are also produced by neutrophils that are increased in photo damaged skin and contribute to the overall pro-oxidant state. Superoxide dismutase (SOD) converts superoxide anion to hydrogen peroxide. Hydrogen peroxide is able to cross cell membranes easily and in conjunction with Fe²⁺ generates highly toxic hydroxyl radicals. Both singlet oxygen and hydroxyl radicals can initiate lipid peroxidation.

To counteract the harmful effects of reactive oxygen species (ROS), the skin is equipped with antioxidant defense systems consisting of a variety of low molecular weight antioxidants (i.e., vitamin C, Vitamin E, etc.) and antioxidant defense enzymes (i.e., superoxide dismutase, glutathione peroxidase, and catalase) forming an “antioxidant network.” The antioxidant network is responsible for maintaining equilibrium between pro-oxidants and antioxidants. However, the antioxidant defense can be overwhelmed by increased exposure to exogenous sources of ROS. Such a disturbance of the pro-oxidant/antioxidant balance may result in oxidative damage to lipids, proteins and nucleic acids such as DNA.

Release of Iron and Copper

Iron and copper play ambivalent roles in biology because they are required as cofactors for many biological reactions, however, iron and copper toxicity threatens cellular integrity (Hentze, 1995). Iron has two oxidation states—Fe³⁺ has five unpaired electrons in the 3d orbital whereas Fe²⁺ has four. Copper also has two oxidation states—Cu²⁺ has one unpaired electron and Cu⁺ has no unpaired electrons; hence Cu⁺ is not a radical.

In mammalian cells, the level of iron-storage protein is tightly controlled by the iron-regulatory protein-1 at the post-transcriptional level. This regulation prevents iron from acting as a catalyst in reactions between ROS and biomolecules. Recently, it has been shown that both UV-B and UV-A can generate lipid peroxidation induced by iron (Brenneisen et al., 1998; Pourzand et al., 1999). The iron content is substantially elevated over basal levels in the skin of mice exposed to UV-B irradiation and in the skin of sun-exposed healthy individuals (Bisset et al, 1991, 1992). The underlying mechanism appears to be the UV-B induced formation of superoxide radicals and its attack on ferritin, resulting in the release and mobilization of free iron (Cerutti, 1994). Brenneisen et al. have identified the iron-dependant Fenton reaction and lipid peroxidation as the central mechanisms underlying signal transduction of the UV-B response (Brenneisen, 1998). Singlet oxygen and hydrogen peroxide are presently considered to be the most important reactive oxygen species generated intracellularly by UV-A light promoting biological damage in exposed tissues via iron-catalyzed oxidative stress (Halliwell and Gutteridge, 1992).

Gutteridge et al. have found copper in sweat samples from the arm or trunk of athletes immediately after exercise (Gutteridge, 1985). Arm samples also contained much greater concentrations of iron in the samples before exercise. It is also easy to see from these data how training athletes might become anemic by loss of iron.

Unfortunately, skin does not have any defense against oxidative stress induced by free iron and copper. Application of metal chelators having chelating ability to occupy all the coordination sites in iron and copper may be a route to prevent or reduce oxidative damage to skin (Chaudhuri and Puccetti, 2002). The iron-chelating agents have been shown as protectants against UV-radiation-induced free radical production (Bisset, 1991; Jurkiewicz and Buettner, 2001).

Release of Matrix-Degrading Metalloprotease

Matrix Metalloprotease (MMPs) are enzymes able to degrade most components of the extracellular matrix (ECM). At this time more than 20 different MMPs have been identified and classified (Hoekstra et al., 2001). A few selected MMPs relevant to skin care applications along with their substrates are included in Table 1.

TABLE 1
Selected list of matrix metalloprotease family members

| Group | Descriptive name | n | Principal substrate |
|---|---|---|---|
| Collagenase | Interstitial collagenase | MMP-1 | Fibrillar collagen types |
| Gelatinases | Gelatinase A (72 kDa) | MMP-2 | I, II and III Gelatins, |
| | | | non-fibrillar collagen |
| | | | types IV and V |
| | Gelatinase B (92 kDa) | MMP-9 | Gelatins, non-fibrillar |
| | | | collagen types |
| | | | IV and V |
| Stromelysins | Stromelysin-1 | MMP-3 | Proteoglycans, laminin, |
| | | | fibronectin, non- |
| | | | fibrillar collagens |

The ECM not only provides a supportive function for the development and organization of tissues, but also serves as a physical barrier to limit the migration of most normal cells away from their sites of origin. The ECM is not a homogeneous structure: It can include any of several classes of biomolecules, including structural proteins, such as collagens and elastin; adhesion proteins, including fibronectins, lamninins, and entactin; proteoglycans; and glycosaminoglycans. Further, the precise composition of the ECM varies between tissues, and perhaps even in a cell state-specific manner. This complex mixture does not simply surround cells, hold them together, and provide an environment in which interesting events occur, it also directly or indirectly mediates a number of critical biological processes.

Several studies have shown that both UV-A and UV-B cause 4- to 5-fold increase in the production of MMP-1 and MMP-3 (Brenneisen et al., 1993, 1996). In contrast, the synthesis of tissue inhibitory metalloprotease-1 (TIMP-1), natural inhibitor of matrix metalloprotease, increases only marginally. This imbalance is one of the causes of severe connective tissue damage resulting in photo aging of the skin.

The damage caused by excessive MMP on the ECM proteins do not appear overnight, but are the result of the accumulation of successive molecular damage, especially in the case of overexposure to UV light. The repercussions of the degradation of ECM proteins may then be revealed in many ways depending on age, genetic program, and life-style and of course on the general health status of the individual. Application of MMP inhibitors may be a route to prevent or minimize damage to extracellular matrix proteins (Thibodeau, 2000).

Therefore, in order to reduce or prevent the visible affects caused by UV exposure, it is necessary to inhibit or reduce the activity of MMPs such as MMP-1 and MMP-3, to quench reactive free-radical such as superoxide anions and hydroxyl radicals, and/or to chelate elements such as iron and copper to prevent or reduce oxidative stress to the skin.

Compositions comprising collagenase (MMP-1) inhibitors to treat the skin are known in the art. For example, U.S. Pat. No. 5,614,489 relates to skin treatment compositions containing a collagenase and/or elastase inhibitor and a method for reducing evidence of wrinkles and aging by apply such compositions to the skin. In addition, U.S. Pat. No. 6,365,630 relates to compositions and methods for ameliorating the effects of UV-A and UV-B radiation from the sun. The recited compositions comprise UV-A and UV-B blockers and MMP inhibitors. However, neither of these references provides for the use of compositions based on extracts of Phyllanthus emblica in methods to treat skin conditions associated with aging and exposure to UV radiation.

Furthermore, U.S. Pat. No. 6,362,167 relates to a method for blocking, in an animal, free radical processes by using an antioxidant composition comprising an extract of Phyllanthus emblica. Such free radical processes have been implicated in disorders such as premature aging, aging, and age related diseases. However, this patent does not suggest or provide for a method for treating skin conditions associated with UV-induced aging.

An object of one aspect of this invention is to treat skin conditions associated with UV-induced aging.

An object of another aspect of this invention is to inhibit or decrease the expression or activity of matrix metalloproteases, especially stromelysin 1 (MMP-3) and collagenase (MMP-1).

An object of still another aspect of this invention is to chelate iron and copper so as to protect against UV-induced free radical production.

An object of still another aspect of this invention is to provide methods for prophylactically and/or therapeutically regulating UV-induced skin conditions (especially human facial and body skin), using topical compositions containing low molecular-weight tannins.

An object of still another aspect of this invention is to provide methods for prophylactically and/or therapeutically regulating visible and/or tactile discontinuities in mammalian skin texture, including fine lines, wrinkles, enlarged pores, roughness, dryness and other skin texture discontinuities associated with UV-induced aged skin, using topical compositions containing low molecular-weight tannins.

Upon further study of the application, other objects and advantages of the invention will become apparent.

### BRIEF DESCRIPTION OF THE DRAWINGS

Various features and attendant advantages of the present invention will be more fully appreciated as the same becomes better understood when considered in conjunction with the accompanying drawings, in which like reference characters designate the same or similar parts throughout the several views, and wherein:

FIG. 1 is a bar graph showing the protective effects of a standardized extract of Phyllanthus emblica (20 μg/ml) on skin fibroblast cells against superoxide damage.

FIG. 2 is a graph showing the scavenging efficiency of a standardized extract of Phyllanthus emblica and other antioxidants.

FIG. 3 is a bar graph showing the collagenase (MMP-1) inhibitory activity of a standardized extract of Phyllanthus emblica.

FIG. 4 is a bar graph showing the stromelysin 1 (MMP-3) inhibitory activity of a standardized extract of Phyllanthus emblica.

FIG. 5 is a bar graph showing the inhibitory activity of the standardized extract of Phyllanthus emblica on Collagenase (MMP-1) expression.

FIG. 6 is a bar graph showing the ability of a standardized extract of Phyllanthus emblica (Emblica) and other antioxidants to reduce UV-induced erythema. Protocol A-0.2% Emblica extract and 0.5% Vitamin E showed statistically significant (p<0.05) reduction in erythema. Protocol B-Only 0.2% Emblica extract showed statistically significant (p<0.05) reduction in erythema.

### DETAILED DESCRIPTION OF THE INVENTION

Photo-aging of skin is a complex biological process affecting various layers of the skin with major changes seen in the connective tissue of the dermis. The natural shift toward a more pro-oxidant state in intrinsically aged skin can be significantly enhanced following UV-irradiation.

It has been discovered that an extract, preferably a standardized extract of Phyllanthus emblica (syn. Emblica officinalis), more preferably Phyllanthus emblica fruits, has significant anti-aging properties. The Emblica extract of the invention has been shown to reduce UV-induced erythema and has excellent free-radical quenching ability, chelating ability to iron and copper as well as MMP-1 and MVP-3 inhibitory activity with no pro-oxidation activity. Thus, the extract of the invention can be used in effective methods for regulating or improving the appearance of human skin. A standardized extract of Phyllanthus emblica is described in U.S. Pat. No. 6,649,150 B2 issued Nov. 18, 2003.

The preferred composition used in the present invention comprises an extract of low molecular weight hydrolyzable tannins, about 0.05 to about 40% w/w, preferably about 0.05 to about 5%. More specifically, the composition comprises Emblicanin A, Emblicanin B, Punigluconin, Pedunculagin and a cosmetically or pharmaceutically acceptable carrier (60 to 99.9% w/w). Commercially, the above-described tannin composition can be obtained from a suitable plant source such as Phyllanthus emblica (syn. Emblica officinalis) or other suitable sources.

In French patent 2730408 published Aug. 14, 1996, compositions are proposed based on extracts of fruits among which is Phyllanthus emblica (syn. Emblica officinalis). The composition may be based on a dilute-alcoholic extract obtained from the Phyllanthus emblica or an extract obtained, for example by merely pressing the fruit.

Both the extracts obtained by pressing and the extracts obtained by alcoholic maceration may then be concentrated at a moderate temperature under reduced pressure, preferably less than 50° C., then optionally brought to the dry state by freeze-drying or any other method under reduced pressure and at a temperature that is lower than 50° C. so as to avoid degrading the active ingredients of the fruit.

In this French patent, however, there is no indication of the composition of the extracts. Conversely, in U.S. Pat. No. 6,124,268, Ghosal, issued Sep. 26, 2000 entitled “Natural Oxidant Compositions, Method For Obtaining Same And Cosmetic, Pharmaceutical and Nutritional Formulations Thereof” there is set forth the chemical composition of extracts of Emblica officinalis obtained by extracting the fresh fruit at elevated temperatures, e.g. 70° C., using a very dilute aqueous or alcoholic-water salt solution, e.g. 0.1 to 5%. By this extraction process, in the presence of sodium chloride, for example, hydrolysis of the glycocidic enzymes in the plant is prevented and the product is protected from microbial infestation.

In the Ghosal U.S. Pat. No. 6,124,268, the blend of constituents are described under the name of “CAPROS”, with claim 8, for example, of the patent setting forth the composition as follows: A blend consisting essentially of, by weight, (1) and (2) about 35-55% of the gallic/ellagic acid derivatives of 2-keto-glucono-δlactone; (3) about 4-15% of 2,3-di-O-galloyl-4, 6-(S) -hexahydroxydiphenoylgluconic acid; (4) about 10-20% of 2,3,4,6-bis-(S)-hexahydroxydiphenoyl-D-glucose; (5) about 5-15% of 3′,4′,5,7-tetrahydroxyflavone-3-O-rhamnoglucoside; and (6) about 10-30% of tannoids of gallic/ellagic acid.

The common names of the enumerated compounds are (1) Emblicanin A, (2) Emblicanin B, (3) Punigluconin, (4) Pedunculagin and (5) Rutin.

The preferred composition used in the present invention comprises a modification of the CAPROS composition, comprising a standardized extract of low molecular weight (<1000) hydrolyzable tannins, over 40%, preferably 50-80% w/w of Emblicanin A, Emblicanin B, Pedunculagin, and Punigluconin with no or low levels (<1%, w/w) of Rutin (3′,4′,5,7-tetrahydroxyflavone-3-O-rhamnoglucoside). Such a composition is discussed with greater specificity in pages 28-30 of the August 2001 issue of Soap, Perfumery and Cosmetics, the article having the title Ingredients/Emblica, Bearing Fruit, by Ratan K. Chaudhuri. The preferred concentrations of Rutin in the standardized extract are less than 1.0%, less than 0.01%, less than 0.001% and less than 0.0001%, with a value of 0.01 to 0.001% being particularly preferred. As for the other ingredients, the most preferred concentrations of the other components are on a percent by weight basis of the total dried extract:

| | Most Preferred |
|---|---|
| | Concentrations |
| | % by weight |
| Emblicanin A | 20-35 |
| Emblicanin B | 10-20 |
| Pedunculagin | 15-30 |
| Punigluconin | 3-12 |

The standardized composition preferably exhibits average percentage deviations from these preferred values of:

| | Preferred | Most Preferred |
|---|---|---|
| | Deviation | Deviation |
| Emblicanin A | ±10% | ±5% |
| Emblicanin B | ±10% | ±5% |
| Pedunculagin | ±10% | ±5% |
| Punigluconin | ±10% | ±5% |

The composition can be obtained by removal of Rutin by reversed-phase column chromatography or HPLC using a solvent system of acetonitrile, water/phosphoric acid (20/80/1) or other solvent combinations as it elutes faster than the low molecular-weight tannins. Also, by selection of geographical location, the Phyllanthus emblica fruit extract may provide a substantially lower level of Rutin (<1.0%, w/w). It has been observed that medium-sized fruits collected from some parts of eastern India, during October-November, after water extraction and drying, yielded the preferred composition as a powder with the desired low content of Rutin. Accordingly, by analyzing the Rutin content of extracts and selecting such extracts that contain the desired low content of Rutin, it is possible to prepare a standardized extract.

The resultant standardized extract powdery material is then incorporated in a cosmetically or pharmaceutically acceptable carrier, preferably having a pH ranging from about 3 to 6.5. The carrier is any conventional carrier for topical administration and is preferably employed in a concentration of about 60%-99.9%, preferably 90% to 99.7%, and more preferably 95% to 99.5%. (In other words, the concentration of the composition of the present invention is generally about 0.1 to 40% by weight, preferably 0.3 to 10% by weight, and more preferably 0.5 to 5% by weight.).

The composition can be used to treat or regulate skin conditions characterized as visible and/or tactile discontinuities in the skin, signs of aging, and visible and/or tactile discontinuities in the skin associated with skin aging (e.g., fine lines, wrinkles, surface roughness, dryness and other texture discontinuities associated with aged skin).

In addition to or included with the above mentioned conditions, this composition can be of use for delaying the appearance of fine lines, enhancing extracellular matrix cohesion, reducing the appearance of spider veins, reducing skin redness, improving skin firmness and elasticity, and reducing damage caused by over exposure to the sun.

The composition used in the present invention can be optionally mixed with other suitable skin care agents, either known prior to the present disclosure as well as those which will be invented in the future. For example, the skin care agents, which can be used include but are not limited to conventional skin care excipients as well as additional photoprotective agents and skin lightening agents.

As for the additional photoprotective agents, if sunscreens are added, suitable sunscreens include any agent capable of protecting the skin from UV radiation including, for example, butyl methoxydibenzoylmethane, cinoxate, benzophenone-8, homosalate, menthyl anthranilate, octocrylene, ethyhexyl methoxycinnamate, ethylhexyl salicylate, benzophenone-3, ethylhexyl dimethyl PABA, glyceryl PABA, phenylbenzimidazole sulfonic acid, benzophenone-4, ethyhexyl triazone, diethylhexyl butamido triazone, bisimidazylate etc.

In addition to photoprotective agents, the compositions and formulations of the present invention are effective for skin whitening and can be optionally blended with other skin whitening agents. For example, the skin whitening products which can be combined include but are not limited to cysteine, 4-thioresorcin, 3-aminotyrosine, 5-hydroxy-2-hydroxymethyl-γ-pyridone, fomesjaponicus and ganoderma extracts, kojic acid, glabridin inhibited tyrosinase, glycyrrhizinic acid, hydroquinone-β-glucoside, catharanthus roseus extract, proteoglycans, proteinase inhibitors, oligopeptides, betaines, and methyl 4-benzyloxy-2-hydroxybenzoate and 4-benzyloxy-2-hydroxybenzoic acid.

For the purposes of providing a topical formulation of the composition of the present invention, any of the known topical excipients can be used therewith such as mineral oils, emulsifying agents, preservatives, anti-oxidants, skin penetrants, etc., including but not limited to the various topical excipients which are utilized in U.S. Pat. No. 6,124,268 and the references discussed above. The compositions can be employed as typical topical compositions utilized in the dermatological and cosmetic fields, e.g., lotions, gels, emulsions, sprays, sticky liposome coacervates, etc.

With respect to the amount of the topical composition which is applied to the skin, it should be a sufficient amount and for a sufficient period of time to visibly change or improve the appearance of the skin. On a molecular level, the amount of the topical composition which is applied to the skin should be a sufficient amount and for a sufficient period of time to inhibit or decrease the expression or function of matrix metalloproteases, such as Collagenase (MMP-1) and Stromelysin-1 (MMP-3). Preferably the topical composition contains an amount of about 0.05 to about 5.0% by weight of the composition in a formulated product and preferably for at least about once per day for a period of preferably at least about two weeks.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius; and, unless otherwise indicated, all parts and percentages are by weight.

The chemistry of the Emblica constituents, including structural formulae and supporting analytical data are found in Ghosal S., V. K. Triphati and S. Chauhan, Active Constituents of Emblica officinalis; Part I—The chemistry and antioxidative effects of the new hydrolysable tannins, Emblican A and B, Indian J. of Chem., 35 B, September 1996, pp 941-948.

### EXAMPLES

Product Description & Standardization

The Emblica antioxidant of the invention is extracted from P. emblica fruits using a water-based process (U.S. Pat. No. 6,124,268 and other pending patents). Alternately, juice obtained from P. emblica fruits, can be dried (spray or freeze drying ) directly to obtain the product in the powder form. The Emblica antioxidant extract of the invention has over 40%, preferably from about 60% to about 85%, of the key chemical components.

The purity of the extract can be upgraded in accordance with the teachings of the cross-referenced application Ser. No. 10/660,742 entitled “Process For Enhancing Bioactive Principles of Phyllanthus Emblica Extract. In addition, the formulations can be provided in anhydrous form in accordance with the teachings of cross-referenced application Ser. No. 10/616,494 entitled “Topical Anhydrous Delivery System”.

The low molecular weight (<1,000) hydrolyzable tannins, namely Emblicanin A and Emblicanin B, along with Pedunculagin and Punigluconin are the key ingredients in the instant Emblica antioxidant. In nature, Emblicanin A and Emblicanin B have only been found in P. emblica plants (Ghosal et al, 1996). Emblica antioxidant has been standardized (Monograph on Emblica, Merck KGaA, 2001) by using high performance thin layer chromatography (HPTLC). Alternately, using high performance liquid chromatography (HPLC) the product can be standardized.

The Emblica antioxidant of the invention has been found to have broad-spectrum antioxidant activity, excellent iron- and copper chelating ability, and MMP-1 and MMP-3 inhibitory activity. These multifunctional attributes of the Emblica antioxidant of the invention are elaborated in the following examples.

### Example I

### The Anti-Oxidation Activity of a Standardized Extract of Phyllanthus Emblica

There are dozens of testing methods available for determining ROS quenching ability of a substance. Described here are the results from two tests—Superoxide anion and hydroxyl radical quenching—carried out for the Emblica extract of the invention and other commercially available antioxidants.

Hypoxantine-Xanthine Oxidase Test (Cell Protection Against Superoxide Damage)

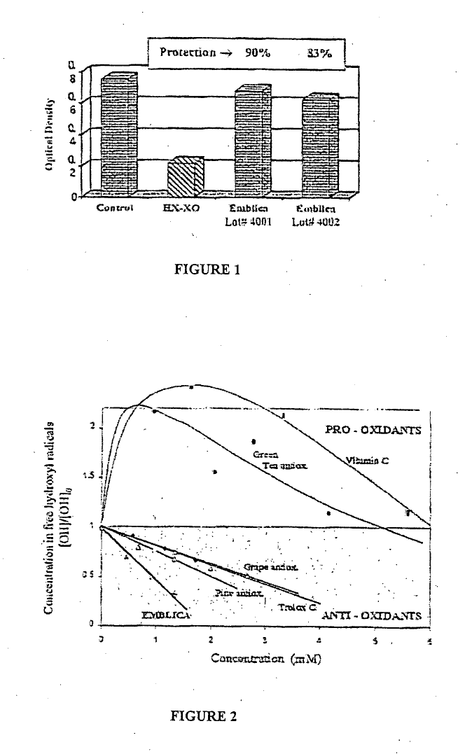

About 90% protection of fibroblast cells against Superoxide damage was observed using 20 μg/ml of the extract of the invention. This study used a human skin fibroblast cell model to determine the cell viability under superoxide [generated by using a hypoxanthine and xanthine oxidase (HX-XO) system (Richard et al, 1992)], and the efficiency of the Emblica extract of the invention to protect cells under this condition. Cell survival was determined with a colorimetric method using 2,3-bis(2-Methoxy-4-nitro-5-sulfophenyl)-5-[9phenylamino)carbonyl]-2H-tetrazolium -hydroxide (XTT). The test is an indication of living cells, which is determined by measuring optical density at 570 nm (FIG. 1).

Deoxyribose Test (Hydroxyl Radical Quenching)

Results demonstrate that the Emblica extract of the invention possesses the strongest hydroxyl radical scavenging ability, significantly better than pine antioxidant, grape antioxidant and trolox C (Vitamin E with the alkyl chain substituted by a carboxylic group). Vitamin C and Green Tea antioxidant are shown to be pro-oxidant under this test system. The method developed by Halliwell et al. was used in this study (Halliwell, 1987).

The hydroxyl radical scavenging efficiency of test products was obtained at respective concentrations of 0.3 mM of FeCl₃, 1.2 mM of EDTA, 33.6 mM of H₂O₂, 33.6 mM of deoxyribose in pH 7.4 phosphate buffer (20 mM) and 0.2-10 mM of chelator. The amount of hydroxyl radicals was determined from the deoxyribose test by using 1% w/v of thiobarbituric acid (TBA) and 2.8% w/v of trichloroacetic acid (TCA). Results are summarized in FIG. 2.

### Example II

### Copper and Iron Chelating Activity of a Standardized Extract of Phyllanthus Emblica

An antioxidant can be a true photoprotective agent provided it chelates the coordination sites in iron and copper (Graf et al, 1984). This is particularly important because the formation of hydroxyl radical from superoxide or hydrogen peroxide from iron requires only one coordination site that is open or occupied by readily dissociable ligand such as water. Water may be completely displaced by stronger ligands such as an azide (N₃⁻) anion. In this Example, the presence of free coordination site(s) in the Fe³⁺-antioxidant complexes were determined by UV spectrophotometric method (Graf et. al., 1984).

Of all the Fe³⁺-chelates tested, only the Emblica-iron (or copper) complex showed the absence of any water coordination. That is, the complex is fully and firmly saturated (i.e., there are no free coordination sites) and there is no room for any pro-oxidant activity via oxo-ferryl or oxo-cupryl radical formation. All other chelators showed disparate coordination site(s) thereby making room for oxo-ferryl or oxo-cupryl radical formation, manifesting pro-oxidant effect, particularly at low concentrations. Tables 2 and 3 below demonstrate that all but the Emblica extract of the invention contained at least one coordinated water molecule. The maximum wavelength, the extinction coefficient of the complex, and the association constant between N₃⁻ and Fe³⁺-antioxidant/chelator complex, are highly variable and depend on the type of bonding between Fe³⁺ and the chelator, the stereochemistry of the complex, and the number of coordination positions.

TABLE 2
Ultraviolet Spectral Data of Fe³⁺ Chelators*

| | Absorption Maxima of | |
|---|---|---|
| | Complex (λₘₐₓ in nm) | |
| Chelator | With Fe³⁺ | N₃ Induced Shift |
| EDTA | 241, 283 | 241, 283, 410 |
| Emblica extract | 241, 294, 353, 377 | 241, 294, 353, 377/No shift |
| Pine Antioxidant | 241, 294, 353, 384 | 241, 294, 353, 400, 440 |
| Vitamin C | 238, 262 | 241, 266, 295 |
| Grape Antioxidant | 247, 295, 353, 396 | 247, 295, 353, 415, 430 |
| Green Tea Antioxidant | 240, 272, 324, 390 | 240, 277, 325, 390 |
| Trolox C | 240, 284 | 240, 273, 284, 360 |
| Gallic Acid | 247, 295, 337 | 247, 295, 353, 412 |

*The peak position were obtained from differential spectroscopic scans of 1.0 mM Fe³⁺ and 5 mM chelator, 1.0M NaN₃ phosphate buffer, pH 7.4, versus the same solution without sodium azide.

TABLE 3
Ultraviolet Spectral Data of Cu²⁺ Chelators*

| | Absorption Maxima of | |
|---|---|---|
| | Complex (λₘₐₓ in nm) | |
| Chelator | With Cu²⁺ | N₃ Induced Shift |
| EDTA | 240, 278 | 241, 279, 354 |
| Emblica extract | 240, 272, 313 | 240, 272, 313/No shift |
| Pine Antioxidant | 239, 279, 302, 331 | 239, 280, 307, 430 |
| Vitamin C | 239, 263 | 239, 263, 284, 364 |
| Grape Antioxidant | 240, 277, 328 | 240, 277, 328, 359 |
| Green Tea Antioxidant | 241, 276, 327, 403 | 240, 277, 336, 404 |
| Trolox C | 241, 288 | 241, 261, 352, 440 |
| Gallic Acid | 240, 258, 321 | 240, 258, 331, 463 |

*The peak position were obtained from differential spectroscopic scans of 1.0 mM Cu²⁺ and 5 mM chelator, 1.0M NaN₃ phosphate buffer, pH 7.4, versus the same solution without sodium azide.

### Example III

### Matrix Metalloprotease (MMP) Inhibitory Activity of a Standardized Extract of Phyllanthus Emblica

Matrix metalloproteases (MMPs, e.g. gelatinase, collagenase and stromelysin), which digest collagen, gelatin (denatured collagen) and other components of ECM, are important for both normal development and carcinogenesis. When cells from one tissue invade a neighboring tissue, as in angiogenesis, wound healing, fetal tissue development and metastasis of tumors, MMPs are released to facilitate the breakdown of barriers opposing the invading cells. There are a number of different MMPs that are specific for the various extracellular matrix (ECM) components. For instance, gelatinise A (MMP-2) is primarily responsible for the degradation of the helical domains of type IV collagen, the principal collagen of basement membranes. Interstitial collagenase (MMP-1) is more selective for type I collagen, while stromelysin 1 (MMP-3) is not so selective as it degrades proteoglycans, laminin, fibronectin and non-fibrillar collagen. A number of inhibitor proteins, termed tissue inhibitors of metalloprotease (TIMPs), and the relationship between MMPs and their respective TIMPs play a key role in the regulation of growth, invasion, and metastasis of neoplastic cells. It has been shown that UV irradiation stimulates the synthesis of MMP-1, MMP-2 and MMP-3 significantly in cultured human fibroblasts, while TIMP synthesis was not altered. Hence unbalanced synthesis of MMPs contributes to the dissolution of dermal and basement membrane compounds finally leading to blister formation and cutaneous photoaging.

Collagenase (MMP-1) Inhibitory Activity

A dose-dependant inhibition of gelatinase/collagenase activity by about 55 to 70% was observed with the Emblica extract of the invention at 150-300 μg/ml.

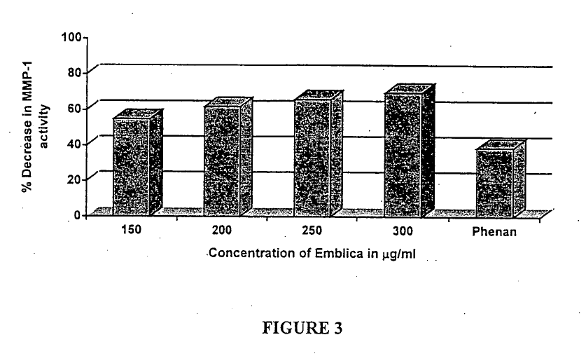

Quantification of gelatinase/collagenase inhibitory activity of the Emblica extract of the invention was determined by using EnzChek® gelatinase/collagenase kit (E-12055) from Molecular Probe by measuring the substrate fluorescence emission at 515 nm. 1,10-Phenanthroline (Phenan) was used as a positive control and collagenase without inhibitor was used as a negative control. Results of this study are summarized in FIG. 3.

Stromelysin-1 (MMP-3) Inhibitory Activity

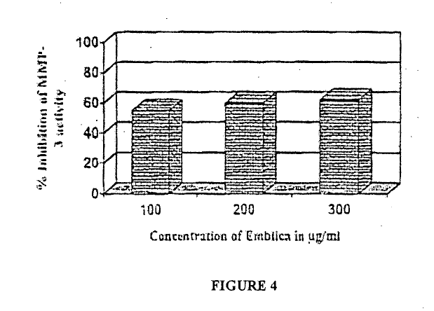

The inhibition of Stromelysin 1 activity by over 50% was observed with the Emblica extract of the invention at 100 μg/ml. Quantification of MMP-3 inhibitory activity of the Emblica extract of the invention was determined by using CHEMICON MMP-3/Stromelysin Activity Assay Kit (ECM 481). The principle of the assay is based upon fluorescent measurement of substrate fragments released upon cleavage of a substrate by MMP-3. Fluorescence intensity of the resulting product is measured and correlated with MMP-3 activity. Results of this study are summarized in FIG. 4.

### Example IV

### Inhibitory Activity of Emblica on Collagenase (MMP-1) Expressions

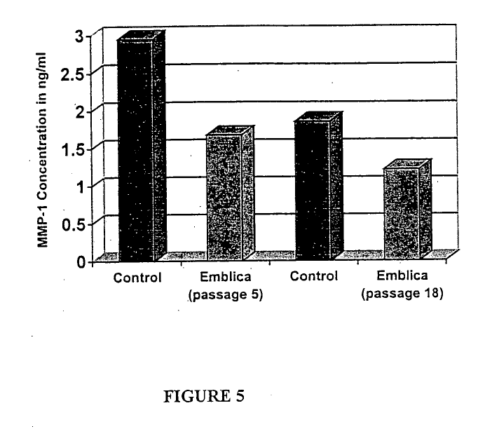

An inhibition of about 40% decrease in collagenase expression was observed using only 50 μg/ml of Emblica. Human skin fibroblast cell lines was used for this study. Quantification of collagenase expression was done by using The Oncogene Research Products MMP-1 ELISA (Catalog # QIA55). This is a non-isotopic immunoassay for the in-vitro quantification of human matrix metalloprotease (MMP-1, interstitial collagenase). Experiments were done using human skin fibroblast cells of fifth and eighteenth passages. Data shows that the effect of Emblica on matrix metalloprotease MMP-1 (collagenase) expression after 48 hrs of incubation in two different experiments is comparable. Results are described in the FIG. 5.

### Example V

### Clinical Study

Erythema, the most familiar manifestation of UV radiation exposure, occurs in a biphasic manner. UV-A mediates the early part of this reaction, known as immediate pigment darkening (IPD) and lasts for about half-hour. Delayed erythema, a function primarily of UV-B dosages, begins 2-8 h after exposure and reaches a maximum in 24-36 h, with erythema, pruritius, and pain in the sun-exposed areas (Lowe and Friedlander, 1997).

Microscopically, changes are detectable as early as 30 min after UV radiation exposure. Epidermal changes include intracellular edema, vacuolization and swelling of melanocytes, and the development of characteristic sunburn cells. In the dermis, UV radiation initially leads to interstitial edema and endothelial cell swelling. Later, there is perivenular edema, degranulation, and loss of mast cells, a decrease in the number of Langerhans cells, neutrophil infiltration, and erythrocyte extravasation (Lowe and Friedlander, 1997).

Protocol

A reduction in UV-induced erythema was used as a criterion for photoprotection and reversal of photo damaged skin. The following protocol was used:

Human volunteers—Eleven (Protocol A)/Ten (Protocol B)

Test sites—Subject's back, 4×2.5 cm area

Test substances—0.2 and 0.5% levels (creams)

Application frequency—2 mg/cm2 once a day

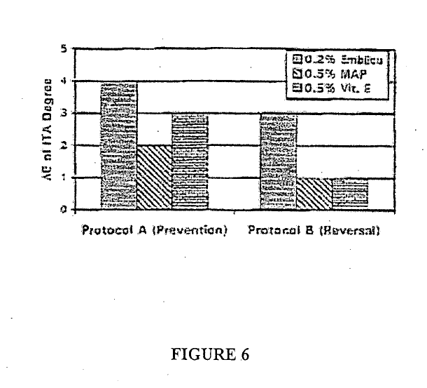

Results—Represented using the individual typology angle (ITA°, COLIPA SPF test method); measured by chromometric measurement. ΔE ITA° was calculated by subtracting treated irradiated site from the untreated irradiated site (FIG. 6).

Definition of ITA Degree—ITA° is calculated using the formula (L*value—Lightness; a*—Color in red-green axis; b*—Color in blue-yellow axis):

ITA°=[Arc Tangent(L*−50)/b*)]180/3.1416

Protocol A (Prevention)—Apply product for 8 days and then induce pigmentation by UV light; compare untreated irradiated control versus the site with product.

Protocol B (Reversal)—Induce pigmentation by UV light; apply product; compare untreated irradiated control to the site with product.

Photo aging of skin is a complex biological process affecting various layers of the skin with major changes seen in the connective tissue of the dermis. The natural shift toward a more pro-oxidant state in intrinsically aged skin can be significantly enhanced following UV-irradiation. The Emblica extract of the invention has been shown to reduce UV-induced erythema and has excellent free-radical quenching ability, chelating ability of iron and copper as well as MMP-1 and MVP-3 inhibitory activity with no pro-oxidation activity.

A properly constituted Phyllanthus emblica extract (Emblica), such as the one described here, may provide a great value as a stand alone photoprotective agent or in combination with sunscreens and/or other anti-aging ingredients for skin care products of the future.

### Example VI

Emblica Formulations
Skin care lotion with 1% Emblica extract

| | TRADE NAME/ | |
|---|---|---|
| INCI NAME | MANUFACTURER | % W/W |
| Phase A | | |
| Water (demineralized) | | 65.97 |
| Disodium EDTA | | 0.10 |
| Propylene Glycol | | 2.00 |
| Sorbitol | Sorbo (70% soln.)/Uniqema | 2.00 |
| Sodium Sauryl Sulfate | Stepanol ME-Dry/Stepan | 0.15 |
| Phase B | | |
| Glyceryl Stearate | Tegin M/Goldschmidt | 5.00 |
| Stearic acid | Emersol 132/Cognis | 1.00 |
| Persea Gratissima (Avocado) oil | Crodarom Avocadin/Croda | 15.00 |
| Unsaponifiables | | |
| Beeswax | White Bleached NF | 1.50 |
| | Beeswax Prills/Ross | |
| Phase C | | |
| Water (demineralized) | | 5.00 |
| Phyllanthus emblica fruit extract | Emblica ™/RONA | 1.00 |
| Phase D | | |
| Triethanolamine | TEA 99%/Union Carbide | 0.28 |
| Phase E | | |
| Propylene glycol, DMDM | Paragon/McIntyre | 1.00 |
| Hydantoin, Methylparaben | | |
| Total | | 100.00 |

Procedure

Combine phase A and heat to 70-75° C. Combine phase B and heat to 70-75° C. Add B to A while stirring. Add phase C at 30° C. Adjust pH to 5.0-6.0 with phase D. Add phase E. Mix until uniform.

Skin Care Lotion with 1% Emblica extract

| | TRADE NAME/ | |
|---|---|---|
| INCI NAME | MANUFACTURER | % W/W |
| Phase A-1 | | |
| Water (demineralized) | | 56.18 |
| Disodium EDTA | | 0.05 |
| Propylene Glycol | | 5.00 |
| Phase A-2 | | |
| Xantham Gum | Vanzan NF/Vanderbilt | 0.25 |
| Magnesium aluminum stearate | Veegum Ultra granules/ | 0.40 |
| | Vanderbilt | |
| Phase B | | |
| Cetearyl alcohol and cetearyl | Montanov 68/Seppic | 7.00 |
| glucoside | | |
| Apricot kernel oil | Lipovol P/Liop | 10.00 |
| Octyl stearate | Cetiol 868/Cognis | 3.00 |
| Dimethicone | Dow Corning 200 fluid 10 | 6.00 |
| | cst/Dow Corning | |
| Phase C | | |
| Water (demineralized) | | 10.00 |
| Phyllanthus emblica fruit extract | Emblica ™/RONA | 1.00 |
| Phase D | | |
| Triethanolamine | TEA 99%/Union Carbide | 0.12 |
| Phase E | | |
| Phenoxyethanol, Isopropylparaben, | Liquapar PE/Sutton | 1.00 |
| Isobutylparaben, butylparaben | | |
| Total | | 100.00 |

Procedure

Disperse phase A-2 in phase A-1 and heat to 70-75° C. Combine phase B and heat to 70-75° C. Add B to A while stirring. Homogenize until mixture cools to 60° C. Add phase C at 30° C. Adjust pH to 4.0-5.0 with phase D. Add phase E. Mix until uniform.

Sunscreen Lotion with 0.1% Emblica extract

| | TRADE NAME/ | |
|---|---|---|
| INCI NAME | MANUFACTURER | % W/W |
| Phase A | | |
| Water (demineralized) | | 66.42 |
| Disodium EDTA | | 0.05 |
| Acrylated/C10-30 Alkyl Acrylates | Carbopol ETD 2020/ | 0.20 |
| Crosspolymer | BF Goodrich | |
| Phase B | | |
| Octyyl Methoxycinnamate | Eusolex 2292/RONA | 7.50 |
| Homosalate | Eusolex HMS/RONA | 5.00 |
| Isopropyl Myristate | Emerest 2314/Cognis | 4.00 |
| C12-15 Alkyl Benzoate | Finsolv TN/Finetex | 4.00 |
| Cetyl alcohol | Crodacol C-70/Croda | 1.50 |
| Stereath-2 | Brij 72/Uniqema | 2.00 |
| Steareth-21 | Brij 721/Uniqema | 2.50 |
| Dimethicone | Dow Corning 200 fluid | 0.50 |
| | 100 cst/Dow Corning | |
| Phase C | | |
| Triethanolamine | TEA 99%/Union Carbide | 0.23 |
| Phase D | | |
| Water (demineralized) | | 5.00 |
| Phyllanthus emblica fruit extract | Emblica ™/RONA | 0.10 |
| Phase E | | |
| Phenoxyethanol, Isopropylparaben, | Liquapar PE/Sutton | 1.00 |
| Isobutylparaben, butylparaben | | |
| Total | | 100.00 |

Procedure

Prepare phase A by dispersing Carbopol in water. Heat the dispersion to 70-75° C. Combine phase B and heat to 70-75° C. Add phase B to phase A while stirring. Add phase C. Homogenize until mixture cools to 40°-45° C. At 30° C. add phase D. Mix until uniform. Add phase E. Stir allowing mixture to cool to RT.

Anti-Aging Lotion with 0.5% Emblica extract

| | TRADE NAME/ | |
|---|---|---|
| INCI NAME | MANUFACTURER | % W/W |
| Phase A-1 | | |
| Water (demineralized) | | 59.15 |
| Disodium EDTA | | 0.05 |
| Propylene Glycol | | 5.00 |
| Phase A-2 | | |
| Xantham Gum | Vanzan NE/Vanderbilt | 0.20 |
| Phase B | | |
| PEG-6 stearate, ceteth-20, glyceryl | Tefose 2561/Gattefosse | 10.00 |
| stearate, steareth-20, stearic acid | | |
| Stearic Acid | Emersol 132/Cognis | 1.00 |
| Hydrogenated castor oil | Cutina HR/Cognis | 1.00 |
| Octyldodecyl myristate | M.O.D./Gattefosse | 8.00 |
| Dimethicone | Dow Corning 200, 50 | 4.00 |
| | cst/Dow Corning | |
| Phenyltrimethicone | Dow Corning 556 Wax/ | 2.00 |
| | Dow Corning | |
| Sweet Almond oil | Cropure Almond/Croda | 3.00 |
| Phase C | | |
| Water (demineralized) | | 5.00 |
| Phyllanthus emblica fruit extract | Emblica ™/RONA | 0.50 |
| Phase D | | |
| Triethanolamine | TEA 99%/Union Carbide | 0.10 |
| Phase E | | |
| Phenoxyethanol, Isopropylparaben, | Liquapar PE/Sutton | 1.00 |
| Isobutylparaben, butylparaben | | |
| Total | | 100.00 |

Procedure

Disperse phase A-2 in phase A-1 and heat to 70-75° C. Combine phase B and heat to 70-75° C. Add B to A while stirring. Homogenize until mixture cools to 60° C. Add phase C at 30° C. Adjust pH to 5.0-6.0 with phase D. Add phase E. Mix until uniform.

Lotion with 0.5% Emblica extract

| | TRADE NAME/ | |
|---|---|---|
| INCI NAME | MANUFACTURER | % W/W |
| Phase A | | |
| Water (demineralized) | | 66.61 |
| Disodium EDTA | | 0.10 |
| Propylene Glycol | | 2.00 |
| Sorbitol | Sorbo (70% soln.)/Uniqema | 2.00 |
| Sodium Sauryl Sulfate | Stepanol ME-Dry/Stepan | 0.15 |
| Phase B | | |
| Glyceryl Stearate | Tegin M/Goldschmidt | 5.00 |
| Stearic acid | Emersol 132/Cognis | 1.00 |
| Persea Gratissima (Avocado) oil | Crodarom Avocadin/Croda | 15.00 |
| Unsaponifiables | | |
| Beeswax | White Bleached NF | 1.50 |
| | Beeswax Prills/Ross | |
| Phase C | | |
| Water (demineralized) | | 5.00 |
| Phyllanthus emblica fruit extract | Emblica ™/RONA | 0.50 |
| Phase D | | |
| Triethanolamine | TEA 99%/Union Carbide | 0.14 |
| Phase E | | |
| Propylene glycol, DMDM | Paragon/McIntyre | 1.00 |
| Hydantoin, Methylparaben | | |
| Total | | 100.00 |

Procedure

Combine phase A and heat to 70-75° C. Combine phase B and heat to 70-75° C. Add B to A while stirring. Add phase C at 30° C. Adjust pH to 5.0-6.0 with phase D. Add phase E. Mix until uniform.

Broad-Spectrum Sunscreen with 0.5% Emblica
and osmoprotectant (Ectoin)

| INCI NAME | TRADE NAME | % W/W |
|---|---|---|
| Phase A | | |
| Butyl Methoxydibenzoylmethane | Eusolex 9020 | 1.00 |
| Glyceryl Stearate, Cetareth-15 | Tego Care 215, Pellets | 3.00 |
| Decyl Oleate | Cetiol V | 5.00 |
| Isopropyl Palmitate | — | 5.00 |
| Dimethicone | Mirasil DM 350 | 0.50 |
| Stearyl Alcohol | Lanette 18 | 2.00 |
| Carbomer | Carbopol ETD 2050 | 0.10 |
| Phase B | | |
| Glycerin (about 87%) | Glycerol | 3.00 |
| Ectoin | RonaCare Ectoin | 0.50 |
| Preservative | | q.s. |
| Water, Ethyhexyl methoxycinnamate, | Eusolex UV-Pearls OMC | 15.00 |
| Silica, PVP, Chlorphenesin, BHT | | |
| Water | Water, deminaralized | q.s |
| Phase C | | |
| Phyllanthus emblica fruit extract | Emblica | 0.30 |
| Phase D | | |
| Sodium hydroxide | Sodium hydroxide, | 0.45 |
| | 10% solution | |
| Phase E | | |
| Perfume | Fragrance “Delicat” | 0.20 |
| Total | | 100.00 |

Procedure:

Heat phases A and B separately to 80 C. Stir phase B into phase A. Homonegige. At 30 C, add phase C. Adjust pH value with sodium hydroxide. Finally add phase E to the emulsion.

Anti-aging Lotion with 0.2% Emblica and 3% RonaCare ™
ASCIII ® (Collagen booster)

| | TRADE NAME/ | |
|---|---|---|
| INCI NAME | MANUFACTURER | % w/w |
| Phase A-1 | | |
| Water (demineralized) | | 56.50 |
| Disodium EDTA | | 0.05 |
| Propylene Glycol | | 5.00 |
| Phase A-2 | | |
| Xantham Gum | Vanzan NF/Vanderbilt | 0.20 |
| Phase B | | |
| PEG-6 stearate, ceteth-20, glyceryl | Tefose 2561/ Gattefosse | 10.00 |
| stearate, steareth-20, stearic acid | | |
| Stearic Acid | Emersol 132/Cognis | 1.00 |
| Hydrogenated castor oil | Cutina HR/Cognis | 1.00 |
| Octyldodecyl myristate | M.O.D./Gattefosse | 8.00 |
| Dimethicone | Dow Corning 200, 50 | 4.00 |
| | cst/Dow Corning | |
| Phenyltrimethicone | Dow Corning 556 | 2.00 |
| | Wax/Dow Coning | |
| Sweet Almond oil | Cropure Almond/Croda | 3.00 |
| Phase C | | |
| Water (demineralized) | | 5.00 |
| Phyllanthus emblica fruit extract | Emblica/RONA | 0.20 |
| Phase D | | |
| Triethanolamine | TEA 99%/Union Carbide | 0.05 |
| Phase E | | |
| Water, lecithin, dipalmitoyl | RonaCare ASCIII/Rona | 3.00 |
| hydroxyproline, phenoxyethanol, tall | | |
| oil sterol, linoleic acid, tocopherol, | | |
| sodium ascorbate, methylparaben, | | |
| butylparaben, ethylparaben, | | |
| propylparaben, mannitol | | |
| Phase F | | |
| Phenoxyethanol, Isopropylparaben, | Liquapar PE/Sutton | 1.00 |
| Isobutylparaben, Butylparaben | | |
| Total | | 100.00 |

Procedure

Disperse A-2 in A-1 and heat to 70-75° C. Combine B and heat to 70-75° C. Add B to A while stirring. Homogenize mixture. At 60° C., mix with propeller mixer. At 30° C. add phase C while mixing. Adjust pH with TEA to 5.0-6.0. Add phase E. Mix until uniform.

References

Bissett D. L., R. Chatteijee and D. P. Hannon, Chronic ultraviolet radiation-induced increase in skin iron and the photoprotective effect of topically applied iron chelators, Photochem. Photobiol, 54, 215-223 (1991).

Bissett D. L. and J. F. McBride, Iron content of human epidermis from sun-exposed and non-exposed body sites, J Soc. Cosmet. Chem. 43, 215-217(1992).

Brenneisen P., J. Wenk, L. O. Klotz, M. Wlaschek, K. Brivia, T. Krieg, H. Sies and K. ScharffetterKochanek, Central role of ferrous-ferric iron in the ultraviolet B irradiation-mediated signaling pathway leading to increased interstitial collagenase (matrix-degrading metalloprotease (MMP)-1) and stromelysin-1 (MMP-3) mRNA levels in cultured human dermal fibroblasts, J. Biol_{—}Chem., 273, 52795287 (1998).

Brenneisen P., J. Oh, M. Wlashek, J. Wenk, K. Briviba, C. Hommel, G. Herrmann, H. Sies, K. Scharffetter-Kochanek, Ultraviolet B wavelength dependence for the regulation of two major matrixmetalloproteinases and their inhibitor TRAP-1 in human dermal fbroblasts, Photochem Photobiol, 64(5), 877-885 (1996).

Cerutti P. A., Oxy-radicals and cancer, Lancet, 344, 862-863 (1994).

Chaudhuri R. K. and G. Puccetti, Transition metal-induced oxidation: implications for skin care products, Cosmetics & Toiletries, 117(9), 43-56, (2002).

Chaudhuri R. K, Emblica cascading antioxidant: A novel natural skin care ingredient, Skin Pharmacol Appl Physiol, 15, 374-380, (2002).

Dreher F., and H. Maibach, Protective effects of topical antioxidants in humans, in Oxidants and Antioxidants in Cutaneous Biology, J. Thiel and P. Elsner (eds), Current Problems in Dermatology, 29, 157-164 (2001).

Ghosal S., V. K. Triphati and S. Chauhan, Active constituents of Emblica officinalis; Part 1—The chemistry and antioxidative effects of two new hydrolysable tannins, Emblicanin A and B, Indian J. of Chem., 35B, 941-948 (1996).

Graf E., J. R. Mahoney, R. G. Byrant, and J. W. Eaton, Iron-catalyzed hydroxyl radical formation, Stringent requirement for free iron coordination site, J. Biol. Chem., 259, 3620-3624 (1984).

Gutteridge J. M. D. A Rowley, B. Halliwell, D. F. Cooper, D. M. Heeley, Copper and iron complexes catalytic for oxygen radical reactions in sweat from human athletes, Clin. Chim Acta, 145, 267-273 (1985).

Halliwell B. and J. M. Gutteridge, Biologically relevant metal ion-dependant hydroxyl radical generation: An Update, FEBSLett, 307, 108-112 (1992).

Halliwell B., J. M. Gutteridge, 01 Aruoma, The deoxyribose method: a simple “test-tube” assay for determination of rate constants for reactions of hydroxyl radicals, Anal Biochem, 165, 215-219 (1987).

Hanson, K M and Simon, J D, Epidermal trans-uronic acid and the UV-A induced photoaging of the skin, Proc. Natl. Acad. Sci. USA, 95, 10576 (1998).

Henize M W, Inducible gene expression (Baeuerle, P A, ed), Birkhauser, Boston, Vol 1, 241-265 (1995).

Hermann G., M. Wlaschek, T. S. Lange, K. Prenzel, G. Goerz, K. Scharffetter-Kochanek, UV-A irradiation stimulates the synthesis of various matrix-metalloproteinases (NEMPs) in cultured human fibroblasts, Exp. Dematol, 2(2), 92-97 (1993)

Hoekstra R., and F. A. L. M. Eskens, J. Verweij, Matrix Metalloprotease Inhibitors: Current Developments and Future Perspectives, The Oncologist, 6, 415-427 (2001).

Jurkiewicz B A and G. R. Buettner, Ultraviolet-light-induced free radical formation in skin: an electron paramagnetic resonance study. Phorchem. Photobiol., 59, 1-4 (1994).

Jurkiewicz Lange B. A. and G. R. Buettner, Electron paramagnetic resonance detection of free radicals in UV-irradiated human and mouse skin, in Oxidants and Antioxidants in Cutaneous Biology, J. Thiele and P. Elsner, eds, in Current Problems in Cutaneous Biology, Basel, Karger, 29, 18-25, (2001).

Lowe N. J. and J. Friedlander, Sunscreens: rationale for use to reduce photodamage and phototoxicity, in Sunscreens, Development. Evaluation and Regulatory Aspects, 2^{nd} edition, N. Lowe, N. Shaath and M. Pathak, eds. Marcel Dekkcr, New York.

Martel A. E., R. Gustafson, and S. Chaberek, in Advances in Catalysis, IX, A. Farakas, Ed., Academic Press, New York, 319 (1957).

Oikarinen A The aging of skin: chronoaging versus photoaging, Phorderm. Photoimmun. Photmed. 43. 34 (1990).

Pourzand, C., R D. Watkin, J. E. Brown, and R. M. Tyrell, Ultraviolet A radiation induces immediate release of iron in human primary skin fibroblasts: The role of ferritin, Proc. Natl. Acad sci., USA, 96, 6751-6756 (1999).

Richard M. J. P. Guiraud, A. M. Monjo and A. Favier, Development of a simple antioxidant screening system assay using human skin fbroblasts, Free Rad Res. Commun, 16(5), 303-314 (1992).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosures of all applications, patents and publications, cited above or below is hereby incorporated by reference.

From the foregoing description, one skilled in the art can easily ascertain the essential characteristics of this invention and, without departing from the spirit and scope thereof, can make various changes and modifications of the invention to adapt it to various usages and conditions.

What is claimed is:

1. A method for regulating the appearance of skin comprising topically applying to said skin a composition comprising:
(a) a cosmetically or pharmaceutically acceptable carrier and about 0.05% to about 5% of an extract comprising a low molecular weight hydrolysable tannins, and mixtures thereof;
(b) an effective amount of at least one additional skin care active ingredient selected from the group consisting of ant-acne actives, retinoids, ant-cellulite agents, antimicrobial actives, antifungal agents, vitamins, anti-inflamatory agents, tanning agents, allantoin, glucosamine, phytantriol, hydroxyacids, niacinamide, phytosterols, sunscreens and mixtures thereof.

2. The method of claim 1, wherein said extract comprises over 40% of Emblicanin A, Emblicanin B, Pedunculagin, and Punigluconin.

3. The method of claim 2, wherein said extract comprises about 60% to about 85% of Emblicanin A, Emblicanin B, Pedunculagin, and Punigluconin.

4. A method for inhibiting or decreasing the expression or function of a matrix metalloprotease in the skin comprising topically applying to said skin a composition comprising a cosmetically or pharmaceutically acceptable carrier and about 0.05% to about 5% of an extract comprising a low molecular weight hydrolysable tannin.

5. The method of claim 4, wherein said extract comprises over about 40% of Emblicanin A, Emblicanin B, Pedunculagin, and Punigluconin.

6. The method of claim 5, wherein said extract comprises about 60% to about 85% of Emblicanin A, Emblicanin B, Pedunculagin, and Punigluconin.

7. A method for inhibiting or decreasing a reactive oxygen species in the skin comprising topically applying to said skin a composition comprising a cosmetically or pharmaceutically acceptable carrier and about 0.05% to about 5% of an extract comprising a low molecular weight hydrolysable tannin.

8. The method of claim 7, wherein said extract comprises over about 40% of Emblicanin A, Emblicanin B, Pedunculagin, and Punigluconin.

9. The method of claim 8, wherein said extract comprises about 60% to about 85% of Emblicanin A, Emblicanin B, Pedunculagin, and Punigluconin.

10. A method for inhibiting or decreasing oxidative stress induced by free iron and copper in the skin comprising topically applying to said skin a composition comprising a cosmetically or pharmaceutically acceptable carrier and about 0.05% to about 5% of an extract comprising a low molecular weight hydrolysable tannin.

11. The method of claim 10, wherein said extract comprises over about 40% of Emblicanin A, Emblicanin B, Pedunculagin, and Punigluconin.

12. The method of claim 11, wherein said extract comprises about 60% to about 85% of Emblicanin A, Emblicanin B, Pedunculagin, and Punigluconin.

13. The method of claim 4, wherein said metalloprotease comprises stromelysin.

14. The method of claim 13, wherein said metalloprotease further comprises collagenase.

15. The method of claim 4, wherein said composition further comprises a UV-A or UV-B sunscreen.

16. The method of claim 4, wherein said composition further comprises another anti-aging ingredient.

17. The method of claim 7, wherein said composition further comprises a UV-A and/or UV-B sunscreen.

18. The method of claim 7, wherein said composition further comprises another anti-aging ingredient.

19. The method of claim 10, wherein said composition further comprises a UV-A and/or UV-B sunscreen.

20. The method of claim 10, wherein said composition further comprises another anti-aging ingredient.
